(19) United States
(12) Patent Application Publication
(10) Pub. No.: US 2007/0089220 A1
(43) Pub. Date: Apr. 26, 2007

(54) Eyeglasses with integral viewing portion

(75) Inventor: Chih-Cheng Shiue, Escondido, CA (US)

Correspondence Address: BRUCE H. TROXELL, SUITE 1404, 5205 LEESBURG PIKE, FALLS CHURCH, VA, 22041, US

(73) Assignee: QDS Injection Molding LLC

(21) Appl. No.: 11/256,145

(22) Filed: Oct. 24, 2005

(51) Int. Cl. A61F 9/02

(52) U.S. Cl. 2/443

(57) ABSTRACT

Provided are eyeglasses such as swim goggles, swim masks, athletic goggles, or safety glasses comprising an integral viewing portion formed of a flexible, transparent material, the viewing portion including one or two frame elements and a viewing member enclosed by the frame element(s) and disposed in front of eyes of a wearer, and a foam flange formed around rear of the viewing member for fitting onto perimeter of the eyes of the wearer; and a flexible strap adapted to attach both ends of the viewing portion to the wearer's head. The invention does not have fragile lenses. Also, its manufacturing processes are simple. Further, it is compact.

### BACKGROUND OF THE INVENTION

2. Field of Invention

The present invention relates to viewing portion of eyeglasses and more particularly to swim goggles, swim masks, athletic goggles, or safety glasses having an integral viewing portion formed of a flexible, transparent material.

2. Related Art

One type of conventional swim goggles comprise a flexible strap, two lenses formed of glass or transparent hard plastics, a foam layer adapted to provide a watertight seal between the lenses and the face of the swimmer, and a flexible flange adapted to secure the lenses and the foam layer together by molding or adhering. However, the lenses are prone to breaking during use. Further, there is a distance between the lens and the eye of the swimmer. It is understood that the longer of the distance the larger of the lens will be for wide viewing. Thus, prior swim goggles are disadvantageous as detailed in prior art described below.

Taiwanese Utility Model Patent No. 91,212,753 having a Publication No. 576,241 discloses swim goggles in FIGS. 1 and 2. The swim goggles comprise two spaced lenses A, a flexible flange B, and a flexible strap C. The swim goggles have a structure simpler than other available prior swim goggles. A plurality of apertures Aa are formed around edge of each lens A for enhancing the coupling of the lens A and the flange B, thereby providing a watertight seal between the lens A and the flange B.

However, the problem of lens break is still not solved. Also, the watertight seal is not reliable. Further, it is not compact and viewing angle is relatively small. Thus, the need for improvement still exists.

### SUMMARY OF THE INVENTION

It is therefore an object of the present invention to provide a pair of eyeglasses (e.g., swim goggles, swim masks, athletic goggles, or safety glasses) comprising an integral viewing portion formed of a flexible, transparent material (e.g., silicon), the viewing portion including one or two frame elements and a viewing member enclosed by the frame element(s) and disposed in front of eyes of a wearer, and a foam flange formed around rear of the viewing member for fitting onto perimeter of the eyes of the wearer; and a flexible strap adapted to attach both ends of the viewing portion to the wearer's head. The present invention has the following advantages: 1) No lens breaking problem. 2) The problem of having a non-reliable sealing among frame, lens, and foam layer is eliminated (i.e., watertight sealing). 3) Simple manufacturing processes in which frame elements and bridge are formed integrally and thus eliminate the processes of separately forming same and assembling them together or the process of molding. 4) Lenses can be disposed closer to the wearer's eyes with wide viewing angle due to no lens breaking problem. 5) Wearer may feel a degree of comfort when wearing the eyeglasses since it is made of flexible material. 6) The eyeglasses are compact.

In one aspect of the present invention, the viewing member comprises two frame elements each adapted to fit onto perimeter of the eye of the wearer, and a bridge interconnected the frame elements in which the frame elements and the bridge either are made of the same material and are integrally formed or are made of the different materials and are formed by molding.

In another aspect of the present invention, a rigid frame is formed around the integral viewing member, the frame having a coupling member at either end as an endpiece for attaching to the strap. The provision of the frame aims at enhancing the structural strength of the frame elements such that the frame elements are not susceptible to being deformed when a strong external force is exerted thereon.

In yet another aspect of the present invention, the viewing member comprises two frame elements each adapted to fit onto perimeter of the eye of the wearer in which the viewing member further comprises two flat or curved face plates disposed closer to the eyes of the wearer.

In a further aspect of the present invention, the strap either is attached to both ends of the viewing member or is attached to outer ends of two spaced rigid frames each formed around the frame element.

The above and other objects, features and advantages of the present invention will become apparent from the following detailed description taken with the accompanying drawings.

### BRIEF DESCRIPTION OF THE DRAWINGS

FIG. 1 is a perspective view of conventional swim goggles;

FIG. 2 is a broken-away perspective view showing the lens and the flange at either side of the swim goggles in FIG. 1;

FIG. 3 is a perspective view of a first preferred embodiment of swim goggles according to the invention;

FIG. 4 is a transverse sectional view of the integral viewing portion in FIG. 3;

FIG. 5 is a longitudinal sectional view of the integral viewing portion in FIG. 3;

FIG. 6 is a transverse sectional view of the integral viewing portion of swim goggles according to a second preferred embodiment of the invention;

FIG. 7 is a transverse sectional view of the integral viewing portion of swim goggles according to another configuration of the second preferred embodiment in FIG.6;

FIG. 8 is a perspective view of a third preferred embodiment of swim goggles according to the invention;

FIG. 9 is a transverse sectional view of the integral viewing portion in FIG. 8; and

FIG. 10 is a transverse sectional view of the integral viewing portion of swim goggles according to another configuration of the third preferred embodiment in FIG. 8.

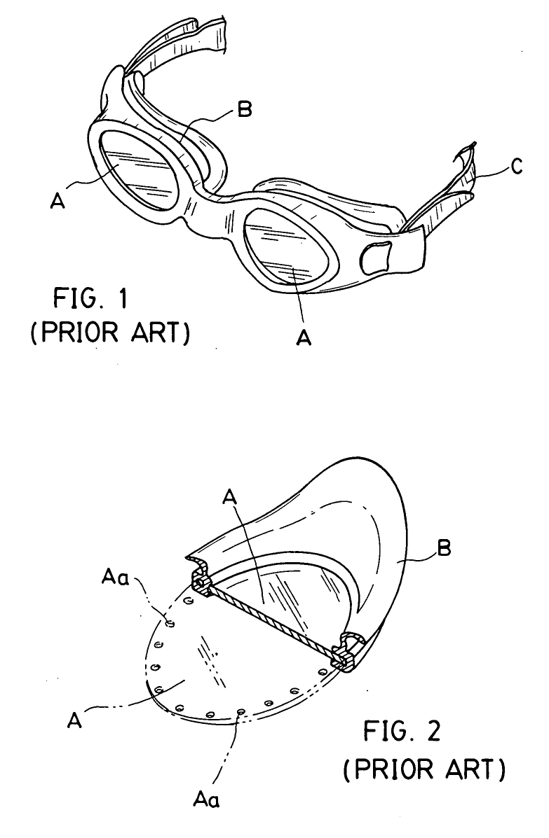

### DETAILED DESCRIPTION OF THE INVENTION

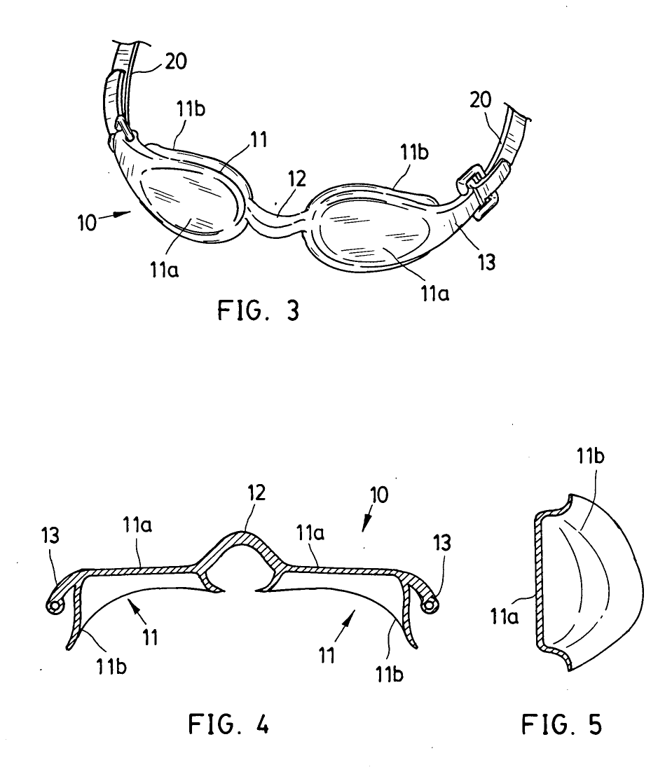

Referring to FIGS. 3, 4, and 5, eyeglasses in accordance with a first preferred embodiment of the invention are shown. The eyeglasses are implemented as swim goggles in preferred embodiments of the invention. It is understood that the eyeglasses may be implemented as swim masks, athletic goggles, or safety glasses in other embodiments without departing from the scope of the invention. The swim goggles comprise an integral viewing portion 10 formed of a flexible, transparent material, and a flexible strap 20 for attaching the goggles to the swimmer's head. The strap 20 is well known in the art and a detailed description thereof is therefore deemed unnecessary.

The viewing portion 10 comprises two frame elements 11, a bridge 12, and two endpieces 13 at both ends of the viewing portion 10 for attaching to the strap 20. The frame element 11 is a closed member adapted to fit onto perimeter of the eye of the swimmer. The frame element 11 is formed of a flexible, transparent material by integrally molding. The material may be silicon or the like. A flat face plate 11a is fitted in the frame element 11. A foam flange 11b is formed around the rear of the frame element 11 for fitting onto perimeter of the eye of the swimmer. The face plate 11a may have a curved section in other embodiments.

The bridge 12 is interconnected the frame elements 11 and is a prior art member. In use the bridge 12 is adapted to extend over an upper portion of the swimmer's nose for holding the frame elements 11 in place. In this embodiment, the frame elements 11 and the bridge 12 are made of the same material and are integrally formed. In other embodiments, the frame elements 11 and the bridge 12 are made of the different materials and are formed by molding. Alternatively, the frame elements 11 and the bridge 12 are separately formed and then are assembled together. Advantageously, the frame elements 11 formed by any of the above three manufacturing processes are watertight.

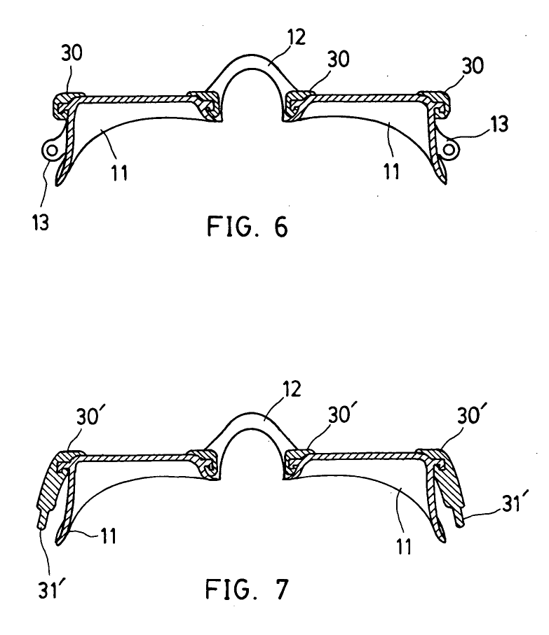

Referring to FIG. 6, a second preferred embodiment of swim goggles according to the invention is shown. A rigid frame 30 is formed around either frame element 11. The provision of the frame 30 aims at enhancing the structural strength of the frame element 11. As such, the frame elements 11 are not susceptible to being deformed when a strong external force is exerted thereon. The bridge 12 may be eliminated if the bridge 12 is integrally formed with the frame 30. That is, only a single frame 30 is provided and it is an integral member.

Referring to FIG. 7, another configuration of the second preferred embodiment of swim goggles is shown. A rigid frame 30′ is formed around either frame element 11. The provision of the frame 30 aims at enhancing the structural strength of the frame element 11. Further, a coupling member 31′ is integrally formed with the frame 30′ and is served as an endpiece for attaching to the strap (not shown). The bridge 12 may be eliminated if the bridge 12 is integrally formed with the frame 30′. That is, only a single frame 30′ is provided and it is an integral member. Note that the frame elements 11 may be formed separately and then are assembled with the frame 30 or 30′ as shown in FIGS. 6 and 7.

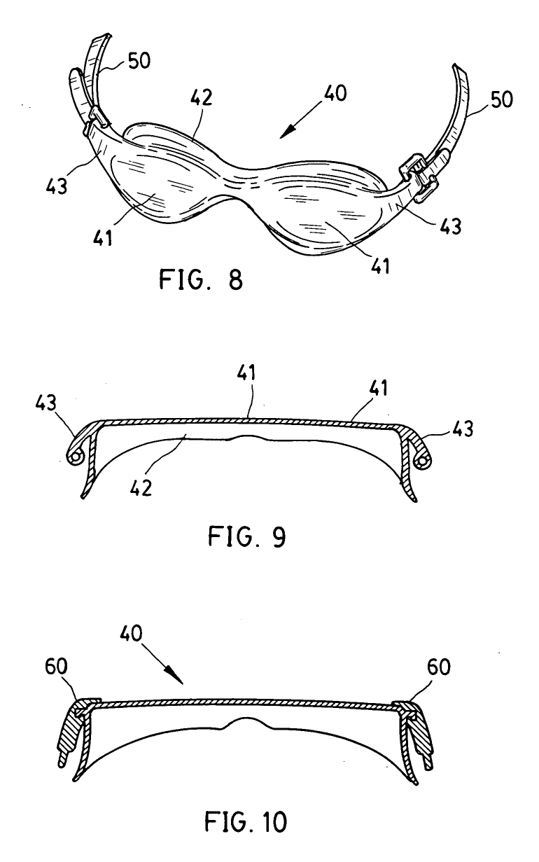

Referring to FIGS. 8 and 9, a third preferred embodiment of swim goggles according to the invention is shown. The frame element 40 is an integral member and comprises a flat, transparent viewing member 41 and a foam flange 42 formed around the viewing member 41. The frame element 40 is adapted to fit onto perimeter of the eyes of the swimmer. Two endpieces 43 are formed at both ends of the viewing member 41 for attaching to the strap 50.

Referring to FIG. 10, another configuration of the third preferred embodiment is shown. A rigid frame 60 is formed around the frame element 40. The provision of the frame 60 aims at enhancing the structural strength of the frame element 40. As such, the frame element 40 is not susceptible to being deformed when a strong external force is exerted thereon. The bridge is eliminated. Also, the frame 60 has two endpieces for attaching to the strap (not shown).

In brief, the frame elements 11 are (or the single frame element 40 is) formed integrally of a flexible, transparent material. Thus, the invention eliminates the prior problems including lens break, and not reliable sealing among frame, lens, and foam layer. Also, the face plates 11a are (or the viewing member 41 is) disposed closer to the eyes of the swimmer, thereby decreasing the size of the viewing portion of the swim goggles and increasing the viewing angle.

While the invention herein disclosed has been described by means of specific embodiments, numerous modifications and variations could be made thereto by those skilled in the art without departing from the scope and spirit of the invention set forth in the claims.

What is claimed is:

1. Eyeglasses comprising:
an integral viewing portion formed of a flexible, transparent material, the viewing portion including a viewing member disposed in front of eyes of a wearer, and a foam flange formed around rear of the viewing member for fitting onto perimeter of the eyes of the wearer; and
a flexible strap adapted to attach both ends of the viewing portion to the wearer's head.

2. The eyeglasses of claim 1, wherein the viewing member comprises two frame elements each adapted to fit onto perimeter of the eye of the wearer.

3. The eyeglasses of claim 2, further comprising a bridge interconnected the frame elements.

4. The eyeglasses of claim 2, wherein the frame elements are formed of transparent silicon.

5. The eyeglasses of claim 3, wherein the bridge is formed of transparent silicon.

6. The eyeglasses of claim 1, wherein the viewing member is flat.

7. The eyeglasses of claim 1, wherein the viewing member is curved.

8. The eyeglasses of claim 1, further comprising a rigid frame formed around the viewing member.

9. The eyeglasses of claim 8, wherein the strap is attached to both ends of the frame.
